(19) United States
(12) Patent Application Publication
(10) Pub. No.: US 2007/0105238 A1
(43) Pub. Date: May 10, 2007

(54) METHOD FOR PROCESSING VALUES FROM A MEASUREMENT

(75) Inventors: Roland Mandl, Ortenburg (DE); Heidi Meyer-Hofer, Ortenburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Micro-Epsilon Messtechnik GmbH & Co.

(21) Appl. No.: 11/619,332

(22) Filed: Jan. 3, 2007

Related U.S. Application Data
(63) Continuation of application No. PCT/DE05/01160, filed on Jul. 1, 2005.

(30) Foreign Application Priority Data
Jul. 6, 2004 (DE) 102004032822.6

(51) Int. Cl. G01N 33/543; G06F 19/00

(52) U.S. Cl. 436/518; 702/19

(57) ABSTRACT

A method for processing values from a measurement in a data set, such that there is recognition of corrupted values from the measurement. The method of the present invention is structured in such a manner that the values from a measurement are compared by means of a suitable measure of difference from a predefinable or determinable or model function and are evaluated via a predefinable or determinable error bound for that measure of difference.

### CROSS REFERENCE TO RELATED APPLICATION

The present application is a continuation of international application PCT/DE 2005/001160, filed 1 Jul. 2005, and which designates the U.S. The disclosure of the referenced application is incorporated herein by reference.

### BACKGROUND OF THE INVENTION

The present invention relates to a method for processing values from a measurement in a data set, such that there is recognition of corrupted values from the measurement.

Methods of this type are known from practice and exist in different developments. Such a method is frequently an automated processing of values from a measurement, where in said processing the recognition and handling of invalid or corrupted values from a measurement in an extensive data set give rise to complications and problems.

Invalid or corrupted values from a measurement can arise due to a plurality of causes. In the simplest case there is an overshoot of the range of measurement, e.g. of a sensor. At the end of the range of measurement, non-linearities or overload effects lead to impermissible values from a measurement. However, other physical effects also lead to an impact on the reliability of measurements. Among these other physical effects are, for example, reflecting or covered points of an object to be measured with, for example, optical triangulation sensors or scanners, or material inhomogeneities in the case of measurements of eddy currents, and so on.

In principle, different cases of errors or the coding of errors in the measurement signal can be distinguished. First, errors can occur that are caused by undershooting or overshooting of the range of measurement. These errors can be recognized by most sensors. These errors occur, for example, if an optical sensor strikes a hole in the object to be measured. This is a so-called out-of-range error.

Furthermore, erroneous measurements or errors can occur that are caused by properties of the object to be measured and can also be interpreted as overshooting or undershooting of the range of measurement. Errors of this type occur, for example, if an optical triangulation sensor is measuring against an extremely dark point of an object to be measured and the amount of light thrown back or reflected is not sufficient for an evaluation. If these errors are recognized by the sensor, then this is usually signaled by an overshooting or undershooting of the range of measurement. This is a so-called poor-target error.

Furthermore, error measurements or errors can occur that are caused by properties of the object to be measured but cannot be recognized as undershooting or overshooting of the range of measurement. For example, erroneous measurements occur in the case of an optical sensor at reflecting or covered points of the object to be measured that are not recognized by the sensor. These erroneous measurements then typically lie within the permitted range of measurement and cannot be distinguished from valid values. Particularly on metallic objects to be measured, reflections occur at corners and grooves, said reflections repeatedly reflecting the laser line in the visible range of the receiver. Furthermore, changes of color in connection with changes in depth of penetration lead to uncertainties in measurement. Surface roughness on the contrary causes a fixed pattern noise due to interference of the laser light. In addition, direct reflections of the laser light to the receiver can also occur at fine grooves, said direct reflections leading to overloading of the matrix.

In an automated evaluation or visualization of values from a measurement it is necessary to eliminate, as nearly as possible, all the values from a measurement that are not correct. In a graphic representation of the values from a measurement in a three-dimensional coordinate system in which the x-y position and the measured height are plotted, the invalid values from a measurement distort the representation, in particular if the height tolerance to be measured is small compared to the range of measurement of the sensor or scanner.

The out-of-range of values from a measurement can in fact be removed from the complete data set in a simple manner but this leads to holes in the surface to be represented in the three-dimensional view. Additional invalid or corrupted values that are caused by properties of the object to be measured cannot be recognized in the case of traditional methods and remain in the representation. A reconstruction of the originally measured surface by interpolation of the missing sampling points can thus lead to an arbitrary erroneous interpretation.

Thus, it is the objective of the present invention to specify a method for processing values from a measurement, where according to said method, reliable recognition of corrupted values from a measurement is made possible.

### SUMMARY OF THE INVENTION

The above objectives and others are realized according to the invention by providing, in one embodiment, a method for processing values from a measurement in a data set, the method comprising comparing values from the measurement by means of a suitable measure of difference from a predefinable or determinable model function and evaluating the values from the measurement via a predefinable or determinable error bound for that measure of difference, thereby recognizing corrupted values from the measurement. According thereto, the method is structured in such a manner that the values from a measurement are compared by means of a suitable measure of difference from a predefinable or determinable model function and are evaluated via a predefinable or determinable error bound for that measure of difference.

In a manner according to the invention it has first been recognized that to recognize corrupted values from a measurement a comparison of the values from a measurement with a model function is particularly suitable. This is a predefinable or determinable model function with which the comparison is done by means of a suitable measure of difference. In so doing, the values from a measurement are evaluated via a predefinable or determinable error bound for the measure of difference. Accordingly, evaluated values from a measurement can then be processed further. Through the comparison according to the invention and the measurement value evaluation with the aid of a model function and an error bound, reliable recognition of corrupted values from a measurement is made possible.

With regard to a particularly reliable recognition of corrupted values from a measurement, the error bound could be determined dynamically from the static distribution of the values from a measurement. In particular, the error bound could be determined via preferably local statistics of the deviations after the removal of the out-of-range values from a measurement. In this connection, a multiple of the standard deviation of the difference between the corrupted values from a measurement and the model function could be used as the error bound. However, it is also conceivable to give the error bound a fixed definition in advance. However, this is disadvantageous in the case of components that lie in an oblique position or that are deformed.

Values from a measurement that overshoot the error bound could be marked as outliers, with a view to additional processing of these values from a measurement. They could, in particular, be values from a measurement that lie outside the range of measurement of a sensor.

Values from a measurement that overshoot the error bound could be removed from the data set as outliers, with a view to realistic visualization of these values from a measurement. Also here, they could, in particular, be values from a measurement that lie outside the range of measurement of a sensor.

In principle, the data set could be in a matrix structure, with a view to a simple visualization of the values from a measurement. In so doing, a visualization as a three-dimensional surface is made possible in a particularly simple manner.

In particular with a view to a simple visualization of the values from a measurement, it is advantageous if the size and/or the type of the data structure in the data set is not changed by the removal of outliers. Furthermore, with a view to a reliable visualization of the values from a measurement, it is advantageous to replace at least one of the outliers by a value from the model function. Thereby a high degree of approximation to the real situation is ensured.

Alternatively thereto, at least one of the outliers could be replaced by a value of the error bound or the maximum deviation between the current data and the model function. Also thereby, a high degree of reality in the imaging is achieved.

As a further alternative, at least one of the outliers could be replaced by an interpolated value. Also thereby, a good approximation to the real situation can be achieved.

With regard to reliable compensation of corrupted values from a measurement, the quality of the model function is of great importance. In this connection, the model function could be adapted in an advantageous manner to the composition and/or geometry of an object to be measured. Along with this, suitable additional knowledge with regard to the structure of the object to be measured can, in an advantageous manner, be introduced in structuring the model function.

The model function could be calculated at sampling points for the reduced values from a measurement. In this case, the reduced values from a measurement are those values from a measurement that have been qualified as reliable and uncorrupted.

With regard to a particularly advantageous model function, that ultimately leads to a particularly reliable recognition of corrupted values from a measurement, an adaptation or re-calculation of the model function and the removal of one or more outliers could be carried out iteratively, where in each step only the outliers with the greatest measure of difference from the model function are removed. By so doing, the model function is optimized incrementally.

Specifically, a multi-dimensional polynomial function could be used as the model function. Model functions of this type have proven themselves particularly suitable.

Furthermore, direct imaging or modeling of the object to be measured could be used to form the model function. Model functions of this type are also advantageous in the framework of the method according to the invention.

In many cases, it could be advantageous that only the deviation from the model function is drawn on for further processing. In other words, addition of the model function in a later step of the method could be omitted by so doing.

The method according to the invention provides reliable recognition of so-called outliers, i.e. impermissible values from a measurement, based on static or dynamic error bounds of a difference function that is determined in comparison to dynamic modeling of the original function, where the original function can be produced from suitable additional knowledge. The recognized outliers can, if necessary, be replaced by accordingly corrected values so that an uncorrupted evaluation of the values from a measurement is made possible.

The method according to the invention is applicable in particular when there is sensor data from arbitrary sensors that can supply information, coded via the output signal, concerning the signal quality or reliability of a measurement. These sensors can,-for example, be optical sensors that are based on the triangulation principle.

In an automated evaluation or visualization of values from a measurement it is advantageous to eliminate, as nearly as possible, all the values from a measurement that are not correct. For this, several problems must be solved. For one thing, the question presents itself of distinguishing correct values from a measurement as against incorrect values from a measurement, so-called outliers. Furthermore, the question presents itself of how the erroneous points can be replaced so that, for example, the visualization is not impacted. Finally, the question presents itself of how the erroneous points can be replaced so that, for example, automated further processing of the values from a measurement is not impacted.

For a realistic representation of the current values from a measurement as a three-dimensional surface, it is necessary to fit the erroneous measurements into the surface so that they are not recognizable as erroneous measurements. In the case of a more extensive evaluation, recognized erroneous measurements should be correspondingly coded and not included in the calculation. Through the replacement of the out-of-range values and the erroneous measurements, an evaluation of the actual values from a measurement is possible.

The method according to the invention is particularly efficient and thus can be used for real-time processing. Along with this, the model function can be adapted to the measurement problem. Furthermore, the error bound can be given a fixed definition in advance or determined dynamically via additional knowledge, e.g. from CAD data.

A subsequent evaluation can be carried out on the corrected original model or on the deviations with respect to the ideal model. In so doing, the regular matrix structure of the values from a measurement remains intact. In comparison to direct interpolation methods, better results are achieved. In addition, the method according to the invention represents an ideal starting basis for interpolation methods since erroneous measurements relating to the object to be measured are also recognized. They can then be replaced by interpolated values, just as in the case of the out-of-range values.

### BRIEF DESCRIPTION OF THE DRAWINGS

Having thus described the invention in general terms, reference will now be made to the accompanying drawings, which are not necessarily drawn to scale, and wherein:

FIG. 1 Shows a flow chart depicting an exemplary embodiment of the method according to the invention;

FIG. 2 Shows a perspective representation of a measurement diagram including corrupted values from a measurement; and

FIG. 3 Shows a perspective representation of a measurement diagram in which the corrupted values from a measurement are replaced by values from the model function.

### DETAILED DESCRIPTION OF THE INVENTION

The present invention now will be described more fully hereinafter with reference to the accompanying drawings, in which some, but not all embodiments of the invention are shown. Indeed, the present invention may be embodied in many different forms and should not be construed as limited to the embodiments set forth herein; rather, these embodiments are provided so that this disclosure will satisfy applicable legal requirements. Like numbers refer to like elements throughout.

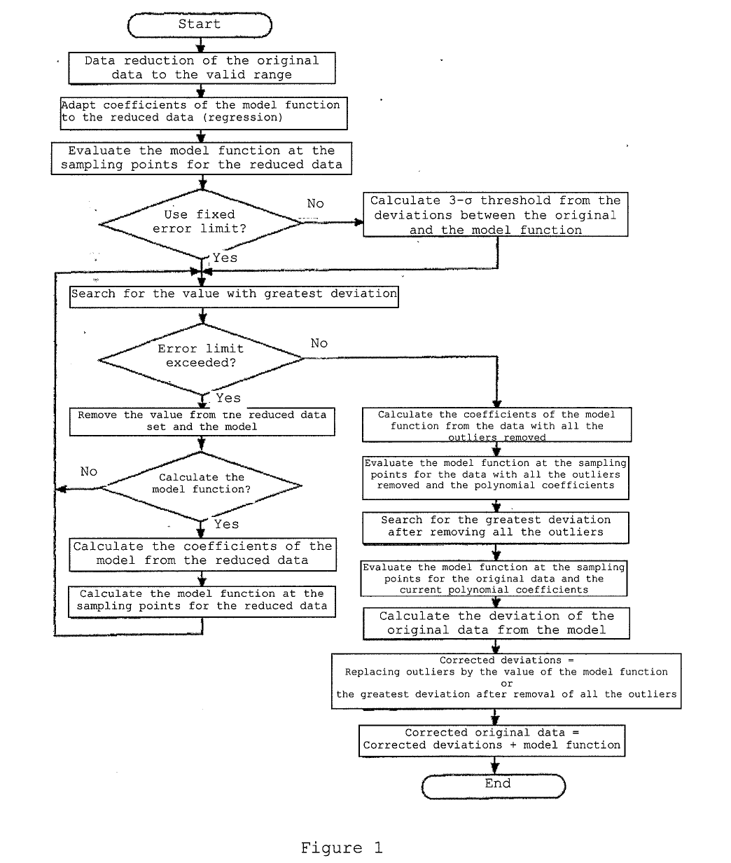

FIG. 1 shows a flow diagram of an exemplary embodiment of the method according to the invention for processing values from a measurement. In the embodiment example of the method according to the invention the out-of-range values are removed from the original data first. Subsequently, the regression coefficients are calculated for the original data with the out-of-range values removed. The model function is evaluated at the sampling points for the reduced original data. Subsequently, erroneous measurements or corrupted values from a measurement are removed until a fixed, predefined error bound or the 3-σ threshold calculated from the deviations between the original data and model function is reached.

In so doing, erroneous measurements or corrupted values from a measurement are repeatedly removed, the coefficients of the model are re-calculated from the reduced data, and the model function is updated at the sampling points for the reduced data.

As soon as the error limit is reached, the updated coefficients, and thereafter the model function at the sampling points for the data cleaned of all the erroneous measurements or corrupted values from a measurement, are re-calculated.

The out-of-range values and erroneous measurements can be replaced either by the values from the model function or by the value of the maximum deviation of the current data from the model. For this, the model function at the sampling points for the original data is calculated with coefficients determined from the reduced data. The deviations between the original data and the model function are re-calculated and the out-of-range values and corrupted values from a measurement or erroneous measurements are corrected accordingly. The corrected original data or original values are calculated from the corrected deviations with the model function's values added thereto.

FIG. 2 shows in a perspective representation the values from a measurement, represented in three-dimensions and including out-of-range values and other corrupted values from a measurement, where a bent, reflecting metal part was measured.

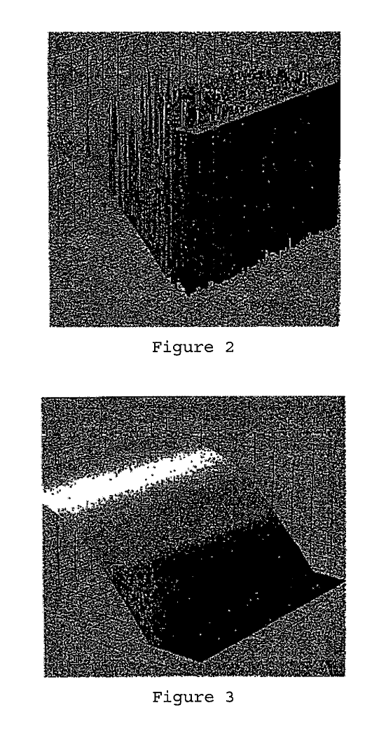

FIG. 3 shows, in a perspective view, a representation of the metal part from FIG. 2 after outliers have been corrected with values from the model function according to the exemplary embodiment of the method for processing values from a measurement. In that view, the bent metal part can be recognized clearly.

If in the method according to the invention the recorded values from a measurement are in a matrix structure, as in the embodiment example shown here, visualization as a three-dimensional surface is possible without further effort. Through the removal of data points from this regular structure a data structure arises that, due to the missing points, can no longer be efficiently processed further or visualized. The method according to the invention has the advantage that the original matrix form of the representation remains intact since the detected erroneous measurements are not removed but rather replaced in a logical manner.

Normally, a fixed error bound for the recognition of outliers is not used since the component measured can lie obliquely or is deformed, that is, can be a free-form surface with deformations. After the component is approximated by a model function, the model function, preferably a three-dimensional model function, can be subtracted from the values from a measurement. After this transformation, error recognition with a constant maximum deviation in form can be used.

Subsequently, all the outliers are replaced, e.g. by the values from the model function at that point. After the model function is once again added to the data from a measurement, the original free-form surface without outliers is obtained once again. Since the calculation of the model function is disturbed by the outliers originally found in the signal, the calculation of the model function and the removal of outliers can be carried out iteratively, where it is always the case that only a small part of the outliers is removed in each step of the calculation.

As the model function, a multi-dimensional polynomial function was used in the example. Any other function that can be approximated to the original curve of the values of the measurement by a linear or non-linear least-squares method can be used just as well. To the extent that there is knowledge concerning the component to be measured, e.g. from CAD data, a direct modeling of the form can be used.

In many cases, it can be logical to omit the addition of the model function in the last step of the method. With this, the more extensive evaluation of the form's deviations from the ideal geometry of the object to be measured, e.g. dents in automobile plating, gap measurements in automobile doors, and so on, can be simplified under certain circumstances.

The error bound for the recognition of outliers can be given a fixed definition in advance or determined via preferably local statistics of the deviations after removal of the out-of-range values. For this, an adjustable multiple of the standard deviation of the difference between the value from a measurement and the model function can be used.

After application of the above method, 3-σ threshold and replacement of the corrupted values from a measurement by the calculated values from the model, a clearly reduced range for the representation of the deviations follows.

With regard to additional advantageous developments and extensions of the method according to the invention, reference is made to the general part of the description as well as to the accompanying claims to avoid repetition.

Finally, let it be expressly pointed out that the exemplary embodiment described above serves merely to explain the claim teaching but does not restrict it to the embodiment example. As such, many modifications and other embodiments of the invention set forth herein will come to mind to one skilled in the art to which this invention pertains having the benefit of the teachings presented in the foregoing descriptions and the associated drawing. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for processing values from a measurement in a data set, said method comprising comparing values from the measurement by means of a suitable measure of difference from a predefinable or determinable model function and evaluating the values from the measurement via a predefinable or determinable error bound for that measure of difference, thereby recognizing corrupted values from the measurement.

2. The method according to claim 1, wherein said measurement is from a sensor measurement.

3. The method according to claim 1, wherein the error bound is determined dynamically from a static distribution of the values from a measurement.

4. The method according to claim 1, wherein a multiple of a standard deviation of a difference between the corrupted values from a measurement and the model function is used as the error bound.

5. The method according to claim 1, wherein values from a measurement that overshoot the error bound are marked as outliers.

6. The method according to claim 5, wherein said outliers are values from a measurement that lie outside a range of measurement of a sensor.

7. The method according to claim 1, wherein the values from a measurement that overshoot the error bound are removed from the data set as outliers.

8. The method according to claim 7, wherein the values from a measurement that overshoot the error bound are values from a measurement that lie outside a range of measurement of a sensor.

9. The method according to claim 1, wherein the data set is present in a matrix structure.

10. The method according to claim 1, wherein a size or a type of a data structure in the data set is not changed by a removal of outliers.

11. The method according to claim 5, wherein at least one of the outliers is replaced with a value from the model function.

12. The method according to claim 5, wherein at least one of the outliers is replaced by a value of the error bound or a maximum deviation of current data from the model function.

13. The method according to claim 5, wherein at least one of the outliers is replaced by an interpolated value.

14. The method according to claim 1, wherein the model function is adapted to a composition or geometry of an object to be measured.

15. The method according to claim 1, wherein the model function is calculated at sampling points for reduced values from a measurement.

16. The method according to claim 1, wherein an adaptation or re-calculation of the model function and removal of one or more outliers are carried out iteratively, and in each step only the outliers with a greatest measure of difference from the model function are removed.

17. The method according to claims 1, wherein a multi-dimensional polynomial function is used as the model function.

18. The method according to claim 1, wherein direct imaging or modeling of an object to be measured is used to form the model function.

19. The method according to claim 1, wherein a deviation from the model function is drawn on for further processing.
